(19) United States
(12) Patent Application Publication
(10) Pub. No.: US 2004/0006797 A1
(43) Pub. Date: Jan. 8, 2004

(54) MYB transcription factors and uses for crop improvement

(76) Inventors: Lifang Shi, St. Charles, MO (US); Stanton B. Dotson, Chesterfield, MO (US); Jingrui Wu, Chesterfield, MO (US); Linda L. Lutfiyya, St. Louis, MO (US); Christine Shewmaker, Woodland, CA (US); Alison Van Eenennaam, Davis, CA (US); Eric D. Aasen, Woodland, CA (US); Diane Ruezinsky, Woodland, CA (US)

Correspondence Address: MONSANTO COMPANY, 800 N. LINDBERGH BLVD., ATTENTION: G.P. WUELLNER, IP PARALEGAL, (E2NA), ST. LOUIS, MO, 63167, US

(21) Appl. No.: 10/407,920

(22) Filed: Apr. 4, 2003

Related U.S. Application Data
(60) Provisional application No. 60/370,759, filed on Apr. 5, 2002.

(51) Int. Cl. A01H 1/00; A01H 5/00; C12N 15/82; C12N 5/04

(52) U.S. Cl. 800/287; 435/69.1; 435/320.1; 435/412; 435/419; 800/320.1; 800/320.3

(57) ABSTRACT

Disclosed herein are inventions in the field of plant biochemistry and genetics. More specifically polynucleotides for use in crop improvement are provided, in particular, plant polynucleotides encoding transcription factors and the polypeptides encoded by such polynucleotides are disclosed. Arrays and DNA constructs comprising such polynucleotides, and polypeptides encoded by such polynucleotides and methods of using the novel polynucleotides and other plant polynucleotide homologs are also disclosed. Novel plants and seeds with improved biological characteristics can be obtained by use of said polynucleotides.

### REFERENCE TO RELATED APPLICATIONS

This application claims priority to U.S. Provisional Application No. 60/370,759 filed Apr. 5, 2002, incorporated herein by reference in its entirety.

### INCORPORATION OF SEQUENCE LISTING

The sequences in the enclosed Sequence Listing are identical to the sequences in the Sequence Listing and computer readable form of prior U.S. Provisional Application No. 60/370,759, filed Apr. 5, 2002, which contain the file named “38-10(52703)A_seq_list.ST25.txt” which is 46 kb and created on Apr. 5, 2002 and which is incorporated herein by reference.

### FIELD OF THE INVENTION

Disclosed herein are inventions in the field of plant biochemistry and genetics. More specifically polynucleotides for use in crop improvement are provided, in particular, plant polynucleotides encoding myb transcription factors and the polypeptides encoded by such polynucleotides are disclosed. Also disclosed are arrays and DNA constructs comprising such polynucleotides, and polypeptides encoded by such polynucleotides. Methods of using the novel polynucleotides and other plant polynucleotide homologs for production of transgenic plants and seeds with improved biological characteristics are disclosed.

### BACKGROUND OF THE INVENTION

The ability to develop transgenic plants with improved traits depends in part on the identification of genes that are useful for production of transformed plants for expression of novel polypeptides. In this regard, the discovery of the polynucleotide sequences of such genes, particularly the polypeptide encoding regions of genes, is needed. Molecules comprising such polynucleotides may be used, for example, in DNA constructs useful for imparting unique genetic properties into transgenic plants.

### SUMMARY OF THE INVENTION

The present invention is directed to novel plant genes which encode a single R2 domain myb transcription factor which are useful for expression in transgenic plants to provide improved plants having higher yield, improved drought tolerance and/or elevated seed oil levels. The invention also encompasses the use of the novel genes and plant homologs for production of transgenic plants and seeds to provide plants, particularly crop plants, having improved properties including improved plant yield resulting from increased nitrogen and/or phosphorus use efficiency, improved drought tolerance, and/or increased seed oil levels.

The present invention also provides homologs of genes encoding negative regulators of root hair development as targets for reduced expression and/or mutagenesis.

### BRIEF DESCRIPTION OF THE DRAWINGS

FIG. 1 is an amino acid sequence alignment of myb transcription factors.

FIG. 2 is a map of a construct (pMON65411) for transformation of transgenic plants for expression of a rice G225 homolog.

FIG. 3 provides data from analysis of oil levels in Canola plants transformed with pMON65411.

### DETAILED DESCRIPTION OF THE INVENTION

The present invention provides novel polynucleotides, or nucleic acid molecules, representing plant MYB transcription factor sequences and the polypeptides encoded by such polynucleotides. The polynucleotides and polypeptides of the present invention find a number of uses, for example in recombinant DNA constructs, in physical arrays of molecules, and for use as plant breeding markers. In addition, the nucleotide and amino acid sequences of the polynucleotides and polypeptides find use in computer based storage and analysis systems. Of particular interest is the use of the novel polynucleotides of the present invention and their plant homologs for production of transgenic crop plants having improved properties, such as improved yield, drought tolerance and increased seed oil levels.

Several genes in Arabidopsis have been shown to be involved in root hair initiation and development, including TTG, WER, CPC and GL2. Wada et al. (U.S. Pat. No. 5,831,060) report that Arabidopsis plants transformed with a CPC gene have an increased number of root hairs and a decreased number of hairs on leaves and stems (glabrous phenotype). Pineda et al. (WO 01/36598) report that Arabidopsis plants transformed with G225 (a single myb domain transcription factor identical to the Arabidopsis CPC gene) and G226, a homolog of G225, demonstrate increased tolerance to nitrogen-limited medium. Pineda et al. also report that overexpression of another single domain myb transcription factor homolog of G225, G682, resulted in transgenic Arabidopsis plants with better germination and growth in heat.

The CPC and G225 polynucleotides are identical having a nucleic acid sequence which is provided as SEQ ID NO: 1 and encode the polypeptide having an amino acid sequence which is provided as SEQ ID NO: 13. The G226 polynucleotide has a nucleic acid sequence which is provided as SEQ ID NO: 2 and encodes the polypeptide having an amino acid sequence which is provided as SEQ ID NO: 14. The G682 polynucleotide has a nucleic acid sequence which is provided as SEQ ID NO: 3 and encodes the polypeptide having an amino acid sequence which is provided as SEQ ID NO: 15. The sequence of Arabidopsis thaliana homolog polynucleotides which are useful in the methods and plants of this invention are provided as SEQ ID NO: 7 through SEQ ID NO: 9; the amino acid sequences of the encoded polypeptides are provided as SEQ ID NO: 19 through SEQ ID NO: 21.

The present invention provides novel polynucleotides that are homologs of the single myb domain transcription factors G225, G226 and G682 and the novel polypeptides encoded by these polynucleotides. The nucleic acid sequence of novel soy homolog polynucleotides are SEQ ID NO: 4 through SEQ ID NO:6; and the amino acid sequence of the encoded soy polypeptides are provided as SEQ ID NO: 16 through SEQ ID NO: 18. The nucleic acid sequence of novel rice homolog polynucleotides are SEQ ID NO: 10 and SEQ ID NO: 11; and the amino acid sequence of the encoded rice polypeptides are provided as SEQ ID NO:22 and SEQ ID NO:23. Nucleotide sequence analysis of SEQ ID NOS: 10 and 11 indicates that the sequences are encoded by the same gene and that the cDNA represented as SEQ ID NO: 11 is likely an improperly spliced cDNA. The nucleic acid sequence of a novel corn homolog polynucleotide is SEQ ID NO: 12; and the amino acid sequence of the encoded corn polypeptide is provided as SEQ ID NO:24.

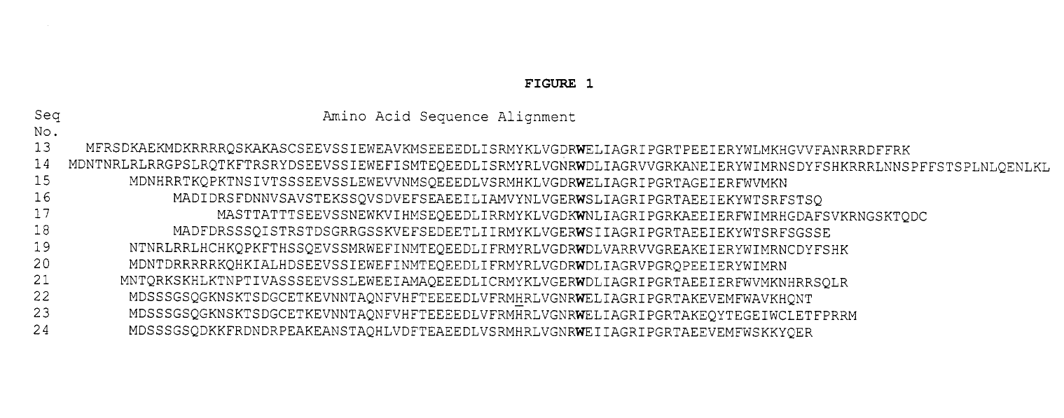

A synthetic consensus amino acid sequence common to the monocot (rice and corn) homologous polypeptides is provided as SEQ ID NO: 25. A synthetic consensus sequence common to the soy homologous polypeptides is provided as SEQ ID NO: 26. A synthetic consensus amino acid sequence common to the Arabidopsis thaliana homologous polypeptides is provided as SEQ ID NO: 27. The consensus sequences were derived by finding regions of common amino acids in SEQ ID NO: 13 through SEQ ID NO: 24 as aligned in FIG. 1.

The present invention also provides methods of using genes involved in root hair development for generation of transgenic plants having improved properties, particularly improved response to nitrogen or phosphorus deficiency, improved growth under drought conditions and/or increased seed oil levels. Of particular interest is the expression in transgenic plants of a single myb domain transcription factor having an amino acid sequence selected from the group consisting of SEQ ID NO: 15 through SEQ ID NO: 27 for production of transgenic plants having improved yield as the result of improved nitrogen utilization. In this case, the term “plants having improved yield” encompasses plants having greater yields as compared to control plants under standard nitrogen fertilization levels, as well as plants which are able to maintain maximum yields when grown under limited nitrogen conditions that cause decreased yields in control plants. Also of interest is the production of transformed plants having improved drought tolerance, improved growth under low levels of phosphorus and/or increased seed oil levels by expression of a myb transcription factor comprising an amino acid sequence selected from the group consisting of SEQ ID NOS: 13-27 or homologs of such sequences.

The effects achieved from expression of the single myb transcription factors and homologs can also be achieved by suppression of genes which encode polypeptides which regulate the root hair development activity of the single myb transcription factors. In this regard the present invention also encompasses the production of transgenic plants having improved nitrogen or phosphorus use, drought tolerance or increased seed oil as described above as the result of decreased expression of other genes involved in root hair development, particularly WEREWOLF (WER) and TTG.

ttg mutant plants have been studied extensively and alter aspects of non-root hair development. Schiefelbein (Plant Physiology (2000) 124:1525-1531) proposes a model in which TTG, a small protein with WD40 repeats, acts at an early stage in epidermis development to activate an R-like bHLH transcription factor, which in turn positively regulates the expression of GL2 to specify the non-hair cell type. In Schiefelbein's proposed regulatory pathway controlling root hair initiation and development, reducing protein activity or expression, e.g. by antisense or knockout, of TTG would disrupt regulation and lead to more root hairs. In the model, the WER protein competes with CPC for interaction with a common bHLH protein and the TTG protein. The complex formed with CPC is unable to activate downstream gene transcription due to CPC having only a single MYB domain. Reducing expression of the WER gene or modifying the WER gene to alter the encoded protein's structure and/or specificity can be used to eliminate competition between WER and CPC. The resulting CPC complex leads to the generation of more root hairs (as in wer mutant plants). The WER gene sequence is available as gi|6601336. The nucleic acid sequence of the cDNA of the WER gene is provided as SEQ ID NO: 28 and the amino acid sequence of the encoded R2R3 myb protein is provided as SEQ ID NO:29. The cDNA of the TTG gene is provided as SEQ ID NO: 30 and the amino acid sequence of the encoded protein is provided as SEQ ID NO: 31.

The present invention provides novel polynucleotides that are homologs of TTG and the novel polypeptides encoded by these polynucleotides. The genomic nucleic acid sequence containing most or all of two novel TTG gene homolog polynucleotides in corn are SEQ ID NO: 32 and SEQ ID NO: 33; and the amino acid sequence of the encoded corn polypeptides are provided as SEQ ID NO: 36 and SEQ ID NO: 37. The genomic nucleic acid sequence containing novel TTG gene homolog polynucleotides in soy are SEQ ID NO: 34 and SEQ ID NO: 35; and the amino acid sequence of the encoded soy polypeptides are provided as SEQ ID NO: 38 and SEQ ID NO: 39.

Depending on the intended use, the polynucleotides of the present invention may be present in the form of DNA, such as cDNA or genomic DNA, or as RNA, for example mRNA. The polynucleotides of the present invention may be single or double stranded and may represent the coding, or sense strand of a gene, or the non-coding, antisense, strand.

The polynucleotides of the present invention find particular use in generation of transgenic plants to provide for increased or decreased expression of the polypeptides encoded by the polynucleotides provided herein. As a result of such biotechnological applications, plants, particularly crop plants, having improved properties are obtained. Crop plants of interest in the present invention include, but are not limited to soy, cotton, canola, maize, wheat, sunflower, sorghum, alfalfa, barley, millet, rice, tobacco, fruit and vegetable crops, and turf grass. Of particular interest are uses of the disclosed polynucleotides to provide plants having improved yield resulting from improved utilization of nitrogen and phosphorous, or resulting from improved responses to drought stress. Also of interest are uses of the polynucleotides to provide transgenic plants having increased seed oil content.

The term “isolated” is used herein in reference to purified polynucleotide or polypeptide molecules. As used herein, “purified” refers to a polynucleotide or polypeptide molecule separated from substantially all other molecules normally associated with it in its native state. More preferably, a substantially purified molecule is the predominant species present in a preparation. A substantially purified molecule may be greater than 60% free, preferably 75% free, more preferably 90% free, and most preferably 95% free from the other molecules (exclusive of solvent) present in the natural mixture. The term “isolated” is also used herein in reference to polynucleotide molecules that are separated from nucleic acids which normally flank the polynucleotide in nature. Thus, polynucleotides fused to regulatory or coding sequences with which they are not normally associated, for example as the result of recombinant techniques, are considered isolated herein. Such molecules are considered isolated even when present, for example in the chromosome of a host cell, or in a nucleic acid solution. The terms “isolated” and “purified” as used herein are not intended to encompass molecules present in their native state.

As used herein a “transgenic” organism is one whose genome has been altered by the incorporation of foreign genetic material or additional copies of native genetic material, e.g. by transformation or recombination.

It is understood that the molecules of the invention may be labeled with reagents that facilitate detection of the molecule. As used herein, a label can be any reagent that facilitates detection, including fluorescent labels (Prober, et al., Science 238:336-340 (1987); Albarella et al., EP 144914), chemical labels (Sheldon et al., U.S. Pat. No. 4,582,789; Albarella et al., U.S. Pat. No. 4,563,417), or modified bases (Miyoshi et al., EP 119448), including nucleotides with radioactive elements, e.g. ³²P, ³³P, ³⁵S or ¹²⁵I such as ³²P deoxycytidine-5′-triphosphate (³²PdCTP).

Polynucleotides of the present invention are capable of specifically hybridizing to other polynucleotides under certain circumstances. As used herein, two polynucleotides are said to be capable of specifically hybridizing to one another if the two molecules are capable of forming an anti-parallel, double-stranded nucleic acid structure. A nucleic acid molecule is said to be the “complement” of another nucleic acid molecule if the molecules exhibit complete complementarity. As used herein, molecules are said to exhibit “complete complementarity” when every nucleotide in each of the molecules is complementary to the corresponding nucleotide of the other. Two molecules are said to be “minimally complementary” if they can hybridize to one another with sufficient stability to permit them to remain annealed to one another under at least conventional “low-stringency” conditions. Similarly, the molecules are said to be “complementary” if they can hybridize to one another with sufficient stability to permit them to remain annealed to one another under conventional “high-stringency” conditions. Conventional stringency conditions are described by Sambrook et al., Molecular Cloning, A Laboratory Manual, 2nd Ed., Cold Spring Harbor Press, Cold Spring Harbor, N.Y. (1989), and by Haynes et al., Nucleic Acid Hybridization, A Practical Approach, IRL Press, Washington, D.C. (1985).

Departures from complete complementarity are therefore permissible, as long as such departures do not completely preclude the capacity of the molecules to form a double-stranded structure. Thus, in order for a nucleic acid molecule to serve as a primer or probe it need only be sufficiently complementary in sequence to be able to form a stable double-stranded structure under the particular solvent and salt concentrations employed. Appropriate stringency conditions which promote DNA hybridization are, for example, 6.0× sodium chloride/sodium citrate (SSC) at about 45° C., followed by a wash of 2.0×SSC at 50° C. Such conditions are known to those skilled in the art and can be found, for example in Current Protocols in Molecular Biology, John Wiley & Sons, N.Y. (1989), 6.3.1-6.3.6. Salt concentration and temperature in the wash step can be adjusted to alter hybridization stringency. For example, conditions may vary from low stringency of about 2.0×SSC at 40° C. to moderately stringent conditions of about 2.0×SSC at 50° C. to high stringency conditions of about 0.2×SSC at 50° C.

As used herein “sequence identity” refers to the extent to which two optimally aligned polynucleotide or peptide sequences are invariant throughout a window of alignment of components, e.g. nucleotides or amino acids. An “identity fraction” for aligned segments of a test sequence and a reference sequence is the number of identical components which are shared by the two aligned sequences divided by the total number of components in the reference sequence segment, i.e. the entire reference sequence or a smaller defined part of the reference sequence. “Percent identity” is the identity fraction times 100. Comparison of sequences to determine percent identity can be accomplished by a number of well-known methods, including for example by using mathematical algorithms, such as those in the BLAST suite of sequence analysis programs.

Polynucleotides—This invention provides polynucleotides comprising regions of cDNAs or genomic DNAs that encode polypeptides. The encoded polypeptides may be the complete protein encoded by the gene or fragments thereof represented by the polynucleotides, or may be fragments of the encoded protein. Preferably, polynucleotides provided herein encode polypeptides constituting a substantial portion of the complete protein, and more preferentially, constituting a sufficient portion of the complete protein to provide the relevant biological activity.

Of particular interest are polynucleotides of the present invention that encode polypeptides involved in one or more important biological functions in plants. Such polynucleotides may be expressed in transgenic plants to produce plants having improved phenotypic properties and/or improved response to stressful environmental conditions.

Polynucleotides of the present invention are generally used to impart such biological properties by providing for enhanced protein activity in a transgenic organism, preferably a transgenic plant, although in some cases, improved properties are obtained by providing for reduced protein expression in a transgenic plant. Reduced protein activity and enhanced protein expression are measured by comparing protein activity with reference to a wild type cell or organism and can be determined by direct or indirect measurement. Direct measurement of protein activity might include an analytical assay for the protein, per se, or enzymatic product of protein activity. Indirect assay might include measurement of a property affected by the protein. Enhanced protein activity can be achieved in a number of ways, for example by overproduction of mRNA encoding the protein or by production of a more active protein using methods such as gene shuffling. One skilled in the are will know methods to achieve overproduction of mRNA, for example by providing increased copies of the native gene or by introducing a construct having a heterologous promoter linked to the gene into a target cell or organism. Reduced protein expression can be achieved by a variety of mechanisms including antisense, mutation or knockout. Antisense RNA will reduce the level of expressed protein resulting in reduced protein activity as compared to wild type activity levels. A mutation in the gene encoding a protein may reduce the level of expressed protein and/or interfere with the function of expressed protein to cause reduced protein activity. Likewise, modification of a gene may alter the encoded protein's secondary structure and/or specificity, e.g. in protein-protein interactions.

A subset of the nucleic molecules of this invention includes fragments of the disclosed polynucleotides consisting of oligonucleotides of at least 15, preferably at least 16 or 17, more preferably at least 18 or 19, and even more preferably at least 20 or more, consecutive nucleotides. Such oligonucleotides are preferably fragments of the larger molecules having a sequence selected from the group of cDNA sequences consisting of SEQ ID NOS: 4, 5, 6, 10, 11 and 12, and find use, for example as probes and primers for detection of the polynucleotides of the present invention.

Also of interest in the present invention are variants of the polynucleotides provided herein. Such variants may be naturally occurring, including homologous polynucleotides from the same or a different species, or may be non-natural variants, for example polynucleotides synthesized using chemical synthesis methods, or generated using recombinant DNA techniques. With respect to nucleotide sequences, degeneracy of the genetic code provides the possibility to substitute at least one base of the protein encoding sequence of a gene with a different base without causing the amino acid sequence of the polypeptide produced from the gene to be changed. Hence, preferred DNA of the present invention may also have any base sequence that has been changed from SEQ ID NOS: 4, 5, 6, 10, 11 and 12 by substitution in accordance with degeneracy of the genetic code. References describing codon usage include: Carels et al., J. Mol. Evol. 46: 45 (1998) and Fennoy et al., Nucl. Acids Res. 21(23):5294 (1993).

Polynucleotides of the present invention that are variants of the polynucleotides provided herein will generally demonstrate significant identity with the polynucleotides provided herein. Of particular interest are polynucleotide homologs having at least about 60% sequence identity, at least about 70% sequence identity, at least about 80% sequence identity, at least about 85% sequence identity, and more preferably at least about 90%, 95% or even greater, such as 98% or 99% sequence identity with polynucleotide sequences described herein.

Protein and Polypeptide Molecules—This invention also provides polypeptides encoded by polynucleotides of the present invention. Amino acid sequences of novel single myb domain polypeptides of the present invention are provided herein as SEQ ID NOS: 16, 17, 18, 22, 23 and 24 and the synthetic consensus sequences of SEQ ID NO: 25 and 26.

As used herein, the term “polypeptide” means an unbranched chain of amino acid residues that are covalently linked by an amide linkage between the carboxyl group of one amino acid and the amino group of another. The term polypeptide can encompass whole proteins (i.e. a functional protein encoded by a particular gene), as well as fragments of proteins. Of particular interest are polypeptides of the present invention which represent whole proteins or a sufficient portion of the entire protein to impart the relevant biological activity of the protein. The term “protein” also includes molecules consisting of one or more polypeptide chains. Thus, a polypeptide of the present invention may also constitute an entire gene product, but only a portion of a functional oligomeric protein having multiple polypeptide chains.

Of particular interest in the present invention are expression of the novel polypeptides, homologous polypeptides provided herein or other homologous polypeptides in transgenic plants to provide plants having improvements in one or more important biological properties, including yield improvement as the result of improved nitrogen or phosphorus utilization, drought tolerance and increased seed oil production. In some cases, decreased expression of polypeptides may be also be desired for obtaining plant improvements, such decreased expression being obtained by use of polynucleotide sequences provided herein, for example in antisense, RNAi or cosuppression methods.

Homologs of the polypeptides of the present invention may be identified by comparison of the amino acid sequence of the polypeptide to amino acid sequences of polypeptides from the same or different plant sources. A variety of homology based search algorithms are available to compare a query sequence to a protein database, including for example, BLAST, FASTA, and Smith-Waterman. A number of values are examined in order to assess the relatedness of the identified homologs. Useful measurements include “E-value” (also shown as “hit_p”), “percent identity”, “percent query coverage”, and “percent hit coverage”.

In BLAST, E-value, or expectation value, represents the number of different alignments with scores equivalent to or better than the raw alignment score, S, that are expected to occur in a database search by chance. The lower the E value, the more significant the match. Because database size is an element in E-value calculations, E-values obtained by BLASTing against public databases, such as GenBank, have generally increased over time for any given query/entry match. Percent identity refers to the percentage of identically matched amino acid residues that exist along the length of that portion of the sequences which is aligned by the BLAST algorithm.

A further aspect of the invention comprises functional homologs which differ in one or more amino acids from those of a polypeptide provided herein as the result of one or more conservative amino acid substitutions. It is well known in the art that one or more amino acids in a native sequence can be substituted with at least one other amino acid, the charge and polarity of which are similar to that of the native amino acid, resulting in a silent change. For instance, valine is a conservative substitute for alanine and threonine is a conservative substitute for serine. Conservative substitutions for an amino acid within the native polypeptide sequence can be selected from other members of the class to which the naturally occurring amino acid belongs. Amino acids can be divided into the following four groups: (1) acidic amino acids, (2) basic amino acids, (3) neutral polar amino acids, and (4) neutral nonpolar amino acids. Representative amino acids within these various groups include, but are not limited to: (1) acidic (negatively charged) amino acids such as aspartic acid and glutamic acid; (2) basic (positively charged) amino acids such as arginine, histidine, and lysine; (3) neutral polar amino acids such as glycine, serine, threonine, cysteine, tyrosine, asparagine, and glutamine; and (4) neutral nonpolar (hydrophobic) amino acids such as alanine, leucine, isoleucine, valine, proline, phenylalanine, tryptophan, and methionine. Conserved substitutes for an amino acid within a native amino acid sequence can be selected from other members of the group to which the naturally occurring amino acid belongs. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Naturally conservative amino acids substitution groups are: valine-leucine, valine-isoleucine, phenylalanine-tyrosine, lysine-arginine, alanine-valine, aspartic acid-glutamic acid, and asparagine-glutamine. A further aspect of the invention comprises polypeptides which differ in one or more amino acids from those of a soy protein sequence as the result of deletion or insertion of one or more amino acids in a native sequence.

Also of interest in the present invention are functional homologs of the polypeptides provided herein which have the same function as a polypeptide provided herein, but with increased or decreased activity or altered specificity. Such variations in protein activity may exist naturally in polypeptides encoded by related genes, for example in a related polypeptide encodes by a different allele or in a different species, or can be achieved by mutagenesis. Naturally occurring variant polypeptides may be obtained by well known nucleic acid or protein screening methods using DNA or antibody probes, for example by screening libraries for genes encoding related polypeptides, or in the case of expression libraries, by screening directly for variant polypeptides. Screening methods for obtaining a modified protein or enzymatic activity of interest by mutagenesis are disclosed in U.S. Pat. No. 5,939,250. An alternative approach to the generation of variants uses random recombination techniques such as “DNA shuffling” as disclosed in U.S. Pat. Nos. 5,605,793; 5,811,238; 5,830,721 and 5,837,458; and International Applications WO 98/31837 and WO 99/65927, all of which are incorporated herein by reference. An alternative method of molecular evolution involves a staggered extension process (StEP) for in vitro mutagenesis and recombination of nucleic acid molecule sequences, as disclosed in U.S. Pat. No. 5,965,408 and International Application WO 98/42832, both of which are incorporated herein by reference.

Polypeptides of the present invention that are variants of the polypeptides provided herein will generally demonstrate significant identity with the polypeptides provided herein. Of particular interest are polypeptides having at least about 35% sequence identity, at least about 50% sequence identity, at least about 60% sequence identity, at least about 70% sequence identity, at least about 80% sequence identity, and more preferably at least about 85%, 90%, 95% or even greater, sequence identity with polypeptide sequences described herein. Of particular interest in the present invention are polypeptides having amino acid sequences provided herein (reference polypeptides) and functional homologs of such reference polypeptides, wherein such functional homologs comprises at least 50 consecutive amino acids having at least 90% identity to a 50 amino acid polypeptide fragment of said reference polypeptide.

Recombinant DNA Constructs—The present invention also encompasses the use of polynucleotides of the present invention in recombinant constructs, i.e. constructs comprising polynucleotides that are constructed or modified outside of cells and that join nucleic acids that are not found joined in nature. Using methods known to those of ordinary skill in the art, polypeptide encoding sequences of this invention can be inserted into recombinant DNA constructs that can be introduced into a host cell of choice for expression of the encoded protein, or to provide for reduction of expression of the encoded protein, for example by antisense or cosupression methods. Potential host cells include both prokaryotic and eukaryotic cells. Of particular interest in the present invention is the use of the polynucleotides of the present invention for preparation of constructs for use in plant transformation.

In plant transformation, exogenous genetic material is transferred into a plant cell. By “exogenous” it is meant that a nucleic acid molecule, for example a recombinant DNA construct comprising a polynucleotide of the present invention, is produced outside the organism, e.g. plant, into which it is introduced. An exogenous nucleic acid molecule can have a naturally occurring or non-naturally occurring nucleotide sequence. One skilled in the art recognizes that an exogenous nucleic acid molecule can be derived from the same species into which it is introduced or from a different species. Such exogenous genetic material may be transferred into either monocot or dicot plants including, but not limited to, soy, cotton, canola, maize, teosinte, wheat, rice and Arabidopsis plants. Transformed plant cells comprising such exogenous genetic material may be regenerated to produce whole transformed plants.

Exogenous genetic material may be transferred into a plant cell by the use of a DNA vector or construct designed for such a purpose. A construct can comprise a number of sequence elements, including promoters, encoding regions, and selectable markers. Vectors are available which have been designed to replicate in both E. coli and A. tumefaciens and have all of the features required for transferring large inserts of DNA into plant chromosomes. Design of such vectors is generally within the skill of the art. See, for example, Plant Molecular Biology: A Laboratory Manual, Clark (ed.), Springier, New York (1997).

A construct will generally include a plant promoter to direct transcription of the protein encoding region or the antisense sequence of choice. Numerous promoters which are active in plant cells have been described in the literature including constitutive promoters, tissue specific promoters and inducible promoters. These include the nopaline synthase (NOS) promoter and octopine synthase (OCS) promoters carried on tumor-inducing plasmids of Agrobacterium tumefaciens, cauliflower mosaic virus promoters such as the cauliflower mosaic virus (CaMV) 19S promoter (Lawton et al., Plant Mol. Biol. 9:315-324 (1987) and 35S promoter (Odell et al., Nature 313:810-812 (1985), CaMV enhanced 35s promoter and the figwort mosaic virus 35S-promoter. Other desirable promoters include the light-inducible promoter from the small subunit of ribulose-1,5-bis-phosphate carboxylase (ssRUBISCO), the actin 1 promoter from rice (McElroy et al. (1991) Mol. Gen. Genet. 231:150-160) or maize (Wang et al. (1992) Molecular and Cellular Biology 12:3399-3406), the Adh promoter (Walker et al., Proc. Natl. Acad. Sci. (U.S.) 84:6624-6628 (1987), the sucrose synthase promoter (Yang et al. (1990) Proc. Natl. Acad. Sci. (U.S.A.) 87:4144-4148), the R gene complex promoter (Chandler et al. (1989) The Plant Cell 1:1175-1183), and the chlorophyll a/b binding protein gene promoter. These promoters and numerous others have been used to create DNA constructs for expression in plants. See, for example, PCT publication WO 84/02913. Any promoter known or found to cause transcription of DNA in plant cells can be used in the invention. Other useful promoters are described, for example, in U.S. Pat. Nos. 5,378,619; 5,391,725; 5,428,147; 5,447,858; 5,608,144; 5,608,144; 5,614,399; 5,633,441; 5,633,435; and 4,633,436, all of which are incorporated herein by reference. Especially preferred promoters include tissue specific promoters such a the root specific promoter disclosed in U.S. Pat. No. 5,837,848, incorporated herein by reference. Especially preferred promoters also include inducible promoters such as cold inducible promoters as disclosed in U.S. Pat. No. 6,084,089, light inducible promoters as disclosed in U.S. Pat. No. 6,294,714, salt inducible promoters as disclosed in U.S. Pat. No. 6,140,078, pathogen inducible promoters as disclosed in U.S. Pat. No. 6,252,138 and phosphorus deficiency inducible promoters as disclosed in U.S. Pat. No. 6,175,060, all of which are incorporated herein by reference.

In addition, promoter enhancers, such as the CaMV 35S enhancer (Kay et al. (1987) Science 236:1299-1302) or a tissue specific enhancer (Fromm et al. (1989) The Plant Cell 1:977-984), may be used to enhance gene transcription levels. Enhancers often are found 5′ to the start of transcription in a promoter that functions in eukaryotic cells, but can often be inserted in the forward or reverse orientation 5′ or 3′ to the coding sequence. In some instances, these 5′ enhancing elements are introns. Deemed to be particularly useful as enhancers are the 5′ introns of the rice actin 1 and rice actin 2 genes. Examples of other enhancers which could be used in accordance with the invention include elements from octopine synthase genes (Ellis et al. (1987) EMBO Journal 6:3203-3208), the maize alcohol dehydrogenase gene intron 1 (Callis et al. (1987) Genes and Develop. 1:1183-1200), elements from the maize shrunken 1 gene, the sucrose synthase intron (Vasil et al. (1989) Plant Physiol. 91:1575-1579) and the TMV omega element (Gallie et al. (1989) The Plant Cell 1:301-311), and promoters from non-plant eukaryotes (e.g., yeast; Ruden et al. (1988) Proc Natl. Acad. Sci. 85:4262-4266).

DNA constructs can also contain one or more 5′ non-translated leader sequences which serve to enhance polypeptide production from the resulting mRNA transcripts. Such sequences may be derived from the promoter selected to express the gene or can be specifically modified to increase translation of the mRNA. Such regions may also be obtained from viral RNAs, from suitable eukaryotic genes, or from a synthetic gene sequence. For a review of optimizing expression of transgenes, see Koziel et al. (1996) Plant Mol. Biol. 32:393-405).

Constructs and vectors may also include, with the coding region of interest, a nucleic acid sequence that acts, in whole or in part, to terminate transcription of that region. One type of 3′ untranslated sequence which may be used is a 3′ UTR from the nopaline synthase gene (nos 3′) of Agrobacterium tumefaciens (Bevan et al. (1983) Nucleic Acids Res. 11:369-385). Other 3′ termination regions of interest include those from a gene encoding the small subunit of a ribulose-1,5-bisphosphate carboxylase-oxygenase (rbcS), and more specifically, from a rice rbcS gene (PCT Publication WO 00/70066), the 3′ UTR for the T7 transcript of Agrobacterium tumefaciens (Dhaese et al. (1983) EMBO J 2:419-426), the 3′ end of the protease inhibitor I or II genes from potato (An et al. (1989) Plant Cell 1:115-122) or tomato (Pearce et al. (1991) Science 253:895-898), and the 3′ region isolated from Cauliflower Mosaic Virus (Timmermans et al. (1990) J Biotechnol 14:333-344). Alternatively, one also could use a gamma coixin, oleosin 3 or other 3′ UTRs from the genus Coix (PCT Publication WO 99/58659).

Constructs and vectors may also include a selectable marker. Selectable markers may be used to select for plants or plant cells that contain the exogenous genetic material. Examples of such include, but are not limited to, a nptII gene (Potrykus et al. (1985) Mol. Gen. Genet. 199:183-188) which codes for kanamycin resistance and can be selected for using kanamycin, G418, etc.; a bar gene which codes for bialaphos resistance; a mutant EPSP synthase gene (Hinchee et al. (1988) Bio/Technology 6:915-922) which encodes glyphosate resistance; a nitrilase gene which confers resistance to bromoxynil (Stalker et al. (1988) J. Biol. Chem. 263:6310-6314); a mutant acetolactate synthase gene (ALS) which confers imidazolinone or sulphonylurea resistance (European Patent Application 154,204 (Sep. 11, 1985)); and a methotrexate resistant DHFR gene (Thillet et al. (1988) J. Biol. Chem. 263:12500-12508.

Constructs and vectors may also include a screenable marker. Screenable markers may be used to monitor transformation. Exemplary screenable markers include a β-glucuronidase or uidA gene (GUS) which encodes an enzyme for which various chromogenic substrates are known (Jefferson (1987) Plant Mol. Biol, Rep. 5:387-405); Jefferson et al. (1987) EMBO J. 6:3901-3907); an R-locus gene, which encodes a product that regulates the production of anthocyanin pigments (red color) in plant tissues (Dellaporta et al. (1988) Stadler Symposium 11:263-282); Other possible selectable and/or screenable marker genes will be apparent to those of skill in the art.

Constructs and vectors may also include a transit peptide for targeting of a gene target to a plant organelle, particularly to a chloroplast, leucoplast or other plastid organelle (European Patent Application Publication Number 0218571).

For use in Agrobacterium mediated transformation methods, constructs of the present invention will also include T-DNA border regions flanking the DNA to be inserted into the plant genome to provide for transfer of the DNA into the plant host chromosome as discussed in more detail below. An exemplary plasmid that finds use in such transformation methods is pCGN8640, a T-DNA vector that can be used to clone exogenous genes and transfer them into plants using Agrobacterium-mediated transformation. pCGN8640 has the restriction sites BamH1, Not1, HindIII, PstII, and SacI positioned between a 35S promoter element and a transcription terminator. Flanking this DNA are the left border and right border sequences necessary for Agrobacterium transformation. The plasmid also has origins of replication for maintaining the plasmid in both E. coli and Agrobacterium tumefaciens strains. A spectinomycin resistance gene on the plasmid can be used to select for the presence of the plasmid in both E. coli and Agrobacterium tumefaciens.

A candidate gene is prepared for insertion into the T-DNA vector, for example using well-known gene cloning techniques such as PCR. Restriction sites may be introduced onto each end of the gene to facilitate cloning. For example, candidate genes may be amplified by PCR techniques using a set of primers. Both the amplified DNA and the cloning vector are cut with the same restriction enzymes, for example, NotI and PstII. The resulting fragments are gel-purified, ligated together, and transformed into E. coli. Plasmid DNA containing the vector with inserted gene may be isolated from E. coli cells selected for spectinomycin resistance, and the presence to the desired insert in pCGN8640 verified by digestion with the appropriate restriction enzymes. Undigested plasmid may then be transformed into Agrobacterium tumefaciens using techniques well known to those in the art, and transformed Agrobacterium cells containing the vector of interest selected based on spectinomycin resistance. These and other similar constructs useful for plant transformation may be readily prepared by one skilled in the art.

Transformation Methods and Transgenic Plants—Methods and compositions for transforming bacteria and other microorganisms are known in the art. See for example Sambrook et al. (1989) Molecular Cloning: A Laboratory Manual, Second Edition, Cold Spring Harbor Laboratory Press, Cold Spring Harbor, N.Y.).

Technology for introduction of DNA into cells is well known to those of skill in the art. Known methods for delivering a gene into cells include: (a) chemical methods (Graham and van der Eb (1973) Virology 54:536-539); (b) physical methods such as microinjection (Capecchi (1980) Cell 22:479-488), electroporation (Wong and Neumann (1982) Biochem. Biophys. Res. Commun. 107:584-587); Fromm et al. (1985) Proc. Natl. Acad. Sci. (U.S.) 82:5824-5828); U.S. Pat. No. 5,384,253); the gene gun (Johnston and Tang (1994) Methods Cell Biol. 43:353-365); (c) viral vectors (Clapp (1993) Clin. Perinatol. 20:155-168); Lu et al. (1993) J. Exp. Med. 178:2089-2096); Eglitis and Anderson (1988) Biotechniques 6:608-614); (d) receptor-mediated mechanisms (Curiel et al. (1992) Hum. Gen. Ther. 3:147-154), Wagner et al. (1992) Proc. Natl. Acad. Sci. (USA) 89:6099-6103); and (e) Agrobacterium tumefaciens-mediated transformation of plants (Fraley et al., Bio/Technology 3:629-635 (1985); and Rogers et al. (1987) Methods Enzymol. 153:253-277). In addition, DNA constructs and methods for stably transforming plant plastids have been described; see, for example U.S. Pat. No. 5,877,402, incorporated herein by reference.

After transformation, the transformed plant cells or tissues may be grown in an appropriate medium to promote cell proliferation and regeneration. In the case of protoplasts the cell wall will first be allowed to reform under appropriate osmotic conditions, and the resulting callus introduced into a nutrient regeneration medium to promote the formation of shoots and roots. For gene gun transformation of wheat and maize see U.S. Pat. Nos. 6,153,812 and 6,160,208, both of which are incorporated herein by reference. See also, Chistou (1996) Particle Bombardment for Genetic Engineering of Plants, Biotechnology Intelligence Unit, Academic Press, San Diego, Calif.), and in particular, pp. 63-69 (maize), and pp50-60 (rice).

The use of Agrobacterium-mediated plant integrating vectors to introduce DNA into plant cells for production of stably transformed whole plants is well known in the art. The region of DNA to be transferred into the host genome is defined by the tDNA border sequences in Agrobacterium-mediated plant integrating vectors and intervening DNA is usually inserted into the plant genome as described (Spielmann et al. (1986) Mol. Gen. Genet. 205:34). See also U.S. Pat. Nos. 5,416,011; 5,463,174; and 5,959,179 for Agrobacterium mediated transformation of soy; U.S. Pat. Nos. 5,591,616 and 5,731,179 for Agrobacterium mediated transformation of monocots such as maize; and U.S. Pat. No. 6,037,527 for Agrobacterium mediated transformation of cotton, all of which are incorporated herein by reference. Modern Agrobacterium transformation vectors are capable of replication in E. coli as well as Agrobacterium, allowing for convenient manipulations (Klee et al. (1985) In: Plant DNA Infectious Agents, Hohn and Schell (eds.), Springer-Verlag, New York, pp. 179-203).

Microprojectile bombardment techniques are also widely applicable, and may be used to transform virtually any plant species. Examples of species which have been transformed by microprojectile bombardment include monocot species such as maize (PCT Publication WO 95/06128), barley, wheat (U.S. Pat. No. 5,563,055), rice, oats, rye, sugarcane, and sorghum, and dicot species including tobacco, soybean (U.S. Pat. No. 5,322,783), sunflower, cotton, tomato, and legumes in general (U.S. Pat. No. 5,563,055).

Any of the polynucleotides of the present invention may be introduced into a plant cell in a permanent or transient manner in combination with other genetic elements such as vectors, promoters enhancers etc. Further any of the polynucleotides of the present invention may be introduced into a plant cell in a manner that allows for production of the polypeptide or fragment thereof encoded by the polynucleotide in the plant cell, or in a manner that provides for decreased expression of an endogenous gene and concomitant decreased production of protein.

It is also to be understood that two different transgenic plants can also be mated to produce offspring that contain two independently segregating added, exogenous genes. Selfing of appropriate progeny can produce plants that are homozygous for both added, exogenous genes that encode a polypeptide of interest. Back-crossing to a parental plant and out-crossing with a non-transgenic plant are also contemplated, as is vegetative propagation.

Expression of the polynucleotides of the present invention and the concomitant production of polypeptides encoded by the polynucleotides is of interest for production of transgenic plants having improved properties, particularly, improved properties which result in crop plant yield improvement. Expression of polypeptides of the present invention in plant cells may be evaluated by specifically identifying the protein products of the introduced genes or evaluating the phenotypic changes brought about by their expression. It is noted that when the polypeptide being produced in a transgenic plant is native to the target plant species, quantitative analyses comparing the transformed plant to wild type plants may be required to demonstrate increased expression of the polypeptide of this invention.

Assays for the production and identification of specific proteins make use of various physical-chemical, structural, functional, or other properties of the proteins. Unique physical-chemical or structural properties allow the proteins to be separated and identified by electrophoretic procedures, such as native or denaturing gel electrophoresis or isoelectric focusing, or by chromatographic techniques such as ion exchange or gel exclusion chromatography. The unique structures of individual proteins offer opportunities for use of specific antibodies to detect their presence in formats such as an ELISA assay. Combinations of approaches may be employed with even greater specificity such as western blotting in which antibodies are used to locate individual gene products that have been separated by electrophoretic techniques. Additional techniques may be employed to absolutely confirm the identity of the product of interest such as evaluation by amino acid sequencing following purification. Although these are among the most commonly employed, other procedures may be additionally used.

Assay procedures may also be used to identify the expression of proteins by their functionality, particularly where the expressed protein is an enzyme capable of catalyzing chemical reactions involving specific substrates and products. These reactions may be measured, for example in plant extracts, by providing and quantifying the loss of substrates or the generation of products of the reactions by physical and/or chemical procedures.

In many cases, the expression of a gene product is determined by evaluating the phenotypic results of its expression. Such evaluations may be simply as visual observations, or may involve assays. Such assays may take many forms including but not limited to analyzing changes in the chemical composition, morphology, or physiological properties of the plant. Chemical composition may be altered by expression of genes encoding enzymes or storage proteins which change amino acid composition and may be detected by amino acid analysis, or by enzymes which change starch quantity which may be analyzed by near infrared reflectance spectrometry.

Suppression of the expression of a transcription factor, e.g. the polynucleotides provided as SEQ ID NO: 29, SEQ ID NO: 31 and homologs including but not limited to SEQ ID NO: 36 through SEQ ID NO: 39, can be achieved by a variety of mechanisms including antisense, cosuppression, ds RNA, mutation or knockout. Antisense, cosuppression and ds RNA mechanisms will reduce the level of protein expressed and the activity will be reduced as compared to wild type expression levels. A mutation in the gene coding for a protein may not decrease the protein expression, but instead interfere with the protein's function to cause reduced protein activity. A knockout can be achieved by homologous recombination with less than the whole gene.

Anti-sense suppression of genes in plants by introducing by transformation of a construct comprising DNA of the gene of interest in an anti-sense orientation is disclosed in U.S. Pat. Nos. 5,107,065; 5,453,566; 5,759,829; 5,874,269; 5,922,602; 5,973,226; 6,005,167; WO 99/32619; WO 99/61631; WO 00/49035; WO 02/02798; all of which are incorporated herein by reference. See also Smith et al. Nature 334: 724-726 (1988), Van der Krol et al., Nature 333: 866-869 (1988), Rothstein et al., Proc. Natl. Aca. Sci. USA 84:8439-8443 (1987), Bird et al., Bio/Technology 9:635-639 (1991), Bartley et al. Biol. Chem. 267:5036-5039 (1992), and Gray et al., Plant Mol. Bio. 19:69-87 (1992).

Co-suppression of genes in a plant by introducing by transformation of a construct for cytoplasmic expression comprising DNA of the gene of interest in a sense orientation is disclosed in U.S. Pat. Nos. 5,034,323; 5,231,020; 5,283,184; 6,271,033, all of which are incorporated herein by reference. See also Krol et al., Biotechniques 6:958-976 (1988), Mol et al., FEBS Lett. 268:427-430 (1990), and Grierson, et al. Trends in Biotech. 9:122-123 (1991).

Interfering RNA suppression of genes in a plant by introducing by transformation of a construct comprising DNA encoding a small (commonly less than 30 base pairs) double-stranded piece of RNA matching the RNA encoded by the gene of interest is disclosed in U.S. Pat. Nos. 5,190,931; 5,272,065; 5,268,149; WO 99/61631; WO 01/75164; WO 01/92513, all of which are incorporated herein by reference.

Processing-defective RNA suppression of genes in a plant by introducing by transformation of a construct comprising DNA encoding a processing-defective copy of the gene of interest in a sense orientation is disclosed in U.S. Pat. No. 5,686,649, incorporated herein by reference.

Transposon tagging genes suppression can be effected by intercrossing a strain with transposons in the locus of the gene of interest with a transposon free strain. See U.S. Pat. No. 6,297,426, incorporated herein by reference.

Backcrossing, using generally accepted plant breeding techniques, can be used to in effect “delete” a native gene. Backcrossing is often used in plant breeding to transfer a specific desirable trait from one inbred or source to an inbred that lacks that trait. This can be accomplished for example by first crossing a superior inbred (A) (recurrent parent) to a donor inbred (non-recurrent parent), which carries a suppressed gene, e.g. a mutant or silenced gene of interest. The progeny of this cross is then mated back to the superior recurrent parent (A) followed by selection in the resultant progeny for the suppressed gene transferred from the non-recurrent parent. After five or more backcross generations with selection for the desired trait, the progeny will be heterozygous for loci controlling the characteristic being transferred, but will be like the superior parent for most or almost all other genes. The last backcross generation would be selfed to give pure breeding progeny for the gene(s) being transferred. A result of any backcrossing method is that the “native” gene is replaced by the suppressed gene.

Transient expression of suppression constructs using viral expression vectors as disclosed in U.S. Pat. No. 6,303,848, incorporated herein by reference, may be a preferred method of gene suppression.

Polynucleotides of the present invention may be used in site-directed mutagenesis. Site-directed mutagenesis may be utilized to modify nucleic acid sequences, particularly as it is a technique that allows one or more of the amino acids encoded by a nucleic acid molecule to be altered (e.g., a threonine to be replaced by a methionine). Three basic methods for site-directed mutagenesis are often employed. These are cassette mutagenesis (Wells et al., Gene 34:315-23 (1985), reference), primer extension (Gilliam et al., Gene 12:129-137 (1980); Zoller and Smith, Methods Enzymol. 100:468-500 (1983); and Dalbadie-McFarland et al., Proc. Natl. Acad. Sci. USA 79:6409-6413 (1982) and methods based upon PCR (Scharf et al., Science 233:1076-1078 (1986); Higuchi et al., Nucleic Acids Res. 16:7351-7367 (1988)). Site-directed mutagenesis approaches are also described in European Patent 0 385 962, European Patent 0 359 472, and PCT Patent Application WO 93/07278.

Post transcriptional gene silencing (PTGS) can result in virus immunity or gene silencing in plants. PTGS is induced by dsRNA and is mediated by an RNA-dependent RNA polymerase, present in the cytoplasm, that requires a dsRNA template. The dsRNA is formed by hybridization of complementary transgene mRNAs or complementary regions of the same transcript. Duplex formation can be accomplished by using transcripts from one sense gene and one antisense gene co-located in the plant genome, a single transcript that has self-complementarity, or sense and antisense transcripts from genes brought together by crossing. The dsRNA-dependent RNA polymerase makes a complementary strand from the transgene mRNA and RNAse molecules attach to this complementary strand (cRNA). These cRNA-RNAse molecules hybridize to the endogene mRNA and cleave the single-stranded RNA adjacent to the hybrid. The cleaved single-stranded RNAs are further degraded by other host RNAses because one will lack a capped 5′ end and the other will lack a poly(A) tail (Waterhouse et al., PNAS 95: 13959-13964 (1998)).

In addition to the above discussed procedures, practitioners are familiar with the standard resource materials which describe specific conditions and procedures for the construction, manipulation and isolation of macromolecules (e.g., DNA molecules, plasmids, etc.), generation of recombinant organisms and the screening and isolating of clones, (see for example, Sambrook et al., Molecular Cloning: A Laboratory Manual, Cold Spring Harbor Press (1989); Mailga et al., Methods in Plant Molecular Biology, Cold Spring Harbor Press (1995; Birren et al., Genome Analysis: Analyzing DNA, 1, Cold Spring Harbor, N.Y. (1998)).

Arrays—The polynucleotide or polypeptide molecules of this invention may also be used to prepare arrays of target molecules arranged on a surface of a substrate. The target molecules are preferably known molecules, e.g. polynucleotides (including oligonucleotides) or polypeptides, which are capable of binding to specific probes, such as complementary nucleic acids or specific antibodies. The target molecules are preferably immobilized, e.g. by covalent or non-covalent bonding, to the surface in small amounts of substantially purified and isolated molecules in a grid pattern. By immobilized is meant that the target molecules maintain their position relative to the solid support under hybridization and washing conditions. Target molecules are deposited in small footprint, isolated quantities of “spotted elements” of preferably single-stranded polynucleotide preferably arranged in rectangular grids in a density of about 30 to 100 or more, e.g. up to about 1000, spotted elements per square centimeter. In addition in preferred embodiments arrays comprise at least about 100 or more, e.g. at least about 1000 to 5000, distinct target polynucleotides per unit substrate. Where detection of transcription for a large number of genes is desired, the economics of arrays favors a high density design criteria provided that the target molecules are sufficiently separated so that the intensity of the indicia of a binding event associated with highly expressed probe molecules does not overwhelm and mask the indicia of neighboring binding events. For high density microarrays each spotted element may contain up to about 10⁷ or more copies of the target molecule, e.g. single stranded cDNA, on glass substrates or nylon substrates.

Arrays of this invention can be prepared with molecules from a single species, preferably a plant species, or with molecules from other species, particularly other plant species. Arrays with target molecules from a single species can be used with probe molecules from the same species or a different species due to the ability of cross species homologous genes to hybridize. It is generally preferred for high stringency hybridization that the target and probe molecules are from the same species.

In preferred aspects of this invention the organism of interest is a plant and the target molecules are polynucleotides or oligonucleotides with nucleic acid sequences having at least 80 percent sequence identity to a corresponding sequence of the same length in a polynucleotide having a sequence selected from the group consisting of SEQ ID NO: 1 through SEQ ID NO: 12 and SEQ ID NO: 28, SEQ ID NO: 30 and SEQ ID NO: 32 through SEQ ID NO: 35 or complements thereof.

Such arrays are useful in a variety of applications, including gene discovery, genomic research, molecular breeding and bioactive compound screening. One important use of arrays is in the analysis of differential gene transcription, e.g. transcription profiling where the production of mRNA in different cells, normally a cell of interest and a control, is compared and discrepancies in gene expression are identified. In such assays, the presence of discrepancies indicates a difference in gene expression levels in the cells being compared. Such information is useful for the identification of the types of genes expressed in a particular cell or tissue type in a known environment. Such applications generally involve the following steps: (a) preparation of probe, e.g. attaching a label to a plurality of expressed molecules; (b) contact of probe with the array under conditions sufficient for probe to bind with corresponding target, e.g. by hybridization or specific binding; (c) removal of unbound probe from the array; and (d) detection of bound probe.

A probe may be prepared with RNA extracted from a given cell line or tissue. The probe may be produced by reverse transcription of mRNA or total RNA and labeled with radioactive or fluorescent labeling. A probe is typically a mixture containing many different sequences in various amounts, corresponding to the numbers of copies of the original mRNA species extracted from the sample.

The initial RNA sample for probe preparation will typically be derived from a physiological source. The physiological source may be selected from a variety of organisms, with physiological sources of interest including single celled organisms such as yeast and multicellular organisms, including plants and animals, particularly plants, where the physiological sources from multicellular organisms may be derived from particular organs or tissues of the multicellular organism, or from isolated cells derived from an organ, or tissue of the organism. The physiological sources may also be multicellular organisms at different developmental stages (e.g., 10-day-old seedlings), or organisms grown under different environmental conditions (e.g., drought-stressed plants) or treated with chemicals.

In preparing the RNA probe, the physiological source may be subjected to a number of different processing steps, where such processing steps might include tissue homogenation, cell isolation and cytoplasmic extraction, nucleic acid extraction and the like, where such processing steps are known to the those of skill in the art. Methods of isolating RNA from cells, tissues, organs or whole organisms are known to those of skill in the art and are described, for example, by Maniatis et al., Molecular Cloning: A Laboratory Manual (Cold Spring Harbor Press) (1989).

Computer Based Systems and Methods

The sequence of the molecules of this invention can be provided in a variety of media to facilitate use thereof. Such media can also provide a subset thereof in a form that allows a skilled artisan to examine the sequences. In a preferred embodiment the polynucleotide and/or the polypeptide sequences of the present invention can be recorded on computer readable media. As used herein, “computer readable media” refers to any medium that can be read and accessed directly by a computer. Such media include, but are not limited to: magnetic storage media, such as floppy discs, hard disc, storage medium, and magnetic tape: optical storage media such as CD-ROM; electrical storage media such as RAM and ROM; and hybrids of these categories such as magnetic/optical storage media. A skilled artisan can readily appreciate how any of the presently known computer readable media can be used to create a manufacture comprising a computer readable medium having recorded thereon a nucleotide sequence of the present invention.

As used herein, “recorded” refers to a process for storing information on computer readable media. A skilled artisan can readily adopt any of the presently known methods for recording information on computer readable media to generate media comprising the nucleotide sequence information of the present invention. A variety of data storage structures are available to a skilled artisan for creating a computer readable medium having recorded thereon a nucleotide sequence of the present invention. The choice of the data storage structure will generally be based on the means chosen to access the stored information. In addition, a variety of data processor programs and formats can be used to store the nucleotide sequence information of the present invention on computer readable media. The sequence information can be represented in a word processing text file, formatted in commercially-available software such as WordPerfect and Microsoft Word, or represented in the form of an ASCII file, stored in a database application, such as DB2, Sybase, Oracle, or the like. A skilled artisan can readily adapt any number of data processor structuring formats (e.g., text file or database) in order to obtain a computer readable medium having recorded thereon the nucleotide sequence information of the present invention.

By providing one or more of polynucleotide or polypeptide sequences of the present invention in a computer readable medium, a skilled artisan can routinely access the sequence information for a variety of purposes. The examples which follow demonstrate how software which implements the BLAST (Altschul et al. (1990) J. Mol. Biol. 215:403-410) and BLAZE (Brutlag et al. (1993) Comp. Chem. 17:203-207) search algorithms on a Sybase system can be used to identify open reading frames (ORFs) within the genome that contain homology to ORFs or polypeptides from other organisms. Such ORFs are polypeptide encoding fragments within the sequences of the present invention and are useful in producing commercially important polypeptides such as enzymes used in amino acid biosynthesis, metabolism, transcription, translation, RNA processing, nucleic acid and a protein degradation, protein modification, and DNA replication, restriction, modification, recombination, and repair.

The present invention further provides systems, particularly computer-based systems, which contain the sequence information described herein. Such systems are designed to identify commercially important fragments of the nucleic acid molecule of the present invention. As used herein, “a computer-based system” refers to the hardware, software, and memory used to analyze the sequence information of the present invention. A skilled artisan can readily appreciate that any one of the currently available computer-based systems are suitable for use in the present invention.

As indicated above, the computer-based systems of the present invention comprise a database having stored therein a nucleotide sequence of the present invention and the necessary hardware and software for supporting and implementing a homology search. As used herein, “database” refers to memory system that can store searchable nucleotide sequence information. As used herein “query sequence” is a nucleic acid sequence, or an amino acid sequence, or a nucleic acid sequence corresponding to an amino acid sequence, or an amino acid sequence corresponding to a nucleic acid sequence, that is used to query a collection of nucleic acid or amino acid sequences. As used herein, “homology search” refers to one or more programs which are implemented on the computer-based system to compare a query sequence, i.e., gene or peptide or a conserved region (motif), with the sequence information stored within the database. Homology searches are used to identify segments and/or regions of the sequence of the present invention that match a particular query sequence. A variety of known searching algorithms are incorporated into commercially available software for conducting homology searches of databases and computer readable media comprising sequences of molecules of the present invention.

Commonly preferred sequence length of a query sequence is from about 10 to 100 or more amino acids or from about 20 to 300 or more nucleotide residues. There are a variety of motifs known in the art. Protein motifs include, but are not limited to, enzymatic active sites and signal sequences. An amino acid query is converted to all of the nucleic acid sequences that encode that amino acid sequence by a software program, such as TBLASTN, which is then used to search the database. Nucleic acid query sequences that are motifs include, but are not limited to, promoter sequences, cis elements, hairpin structures and inducible expression elements (protein binding sequences).

Thus, the present invention further provides an input device for receiving a query sequence, a memory for storing sequences (the query sequences of the present invention and sequences identified using a homology search as described above) and an output device for outputting the identified homologous sequences. A variety of structural formats for the input and output presentations can be used to input and output information in the computer-based systems of the present invention. A preferred format for an output presentation ranks fragments of the sequence of the present invention by varying degrees of homology to the query sequence. Such presentation provides a skilled artisan with a ranking of sequences that contain various amounts of the query sequence and identifies the degree of homology contained in the identified fragment.

Having now generally described the invention, the same will be more readily understood through reference to the following example which is provided by way of illustration, and is not intended to be limiting of the present invention, unless specified.

### EXAMPLE 1

This example illustrates the use of the polynucleotides in providing a desired trait in transgenic plants. Arabidopsis thaliana plants were transformed with vectors comprising a nucleic acid construct comprising a constitutive promoter, CaMV35S, operably linked to one of the polynucleotides selected from SEQ ID NO: 1, 2, 3, 4, 5, 6, 10, 11 and 12. Mutant Arabidopsis thaliana plants having a mutagenized ttgI gene were analyzed as controls. The transgenic and mutagenized plants were grown in a variety of nutrient deficient environments, e.g. low nitrogen, low phosphorus and low water (drought) and analyzed along with appropriate negative control plants to identify transgenic plants having improved properties. Observed physiological phenotypes are reported in Table 1.

TABLE 1

| | more | less | low | low | | | increased |
|---|---|---|---|---|---|---|---|
| SEQ ID | root | antho- | nitrogen | phosphorus | drought | increased | seed |
| NO: | hairs | cyanin | tolerance | tolerance | tolerance | seed oil | protein |
| 1 (G225) | yes | yes | yes | yes | yes | no | no |
| 2 (G226) | yes | yes | yes | yes | yes | yes | no |
| 3 (G682) | yes | yes | yes | yes | yes | no | no |
| 4 (Soy1) | yes | yes | | | | | |
| 5 (Soy2) | yes | yes | yes | | | | |
| 6 (Soy3) | yes | yes | | | | | |
| 10 (Rice1) | yes | yes | yes | | | yes | |
| 11 (Rice2) | no | no | maybe | | | no | |
| 12 (Corn) | no | no | no | | | no | |
| ttgl | yes | yes | yes | | | yes | yes |

Transgenic crop plants expressing G225, G226, G682 or crop gene homologs were generated by transformation of rice to provide for expression of the polypeptides encoded by SEQ ID NOS: 1 and 2, transformation of maize to provide for expression of the polypeptides encoded by SEQ ID NOS: 1, 2, 3, 10, 11 and 12, transformation of soybean to provide for expression of the polypeptides encoded by SEQ ID NOS: 2, 3, 4, 5, 6 and transformation of Brassica napus (Canola) to provide for expression of SEQ ID NO: 2. Expression of the G225 gene or homologs in transgenic plants is under the regulatory control of a CaMV35S promoter.

Preliminary analysis of transgenic maize plants expressing SEQ ID NOS: 1, 10, 11 or 12 indicated that transgenic plants generated with 3 of the 4 recombinant constructs demonstrated a reduced anthocyanin phenotype (lower anthocyanin accumulation in roots, leaf sheath and tassel) similar to the reduced anthocyanin phenotype observed in transgenic Arabidopsis plants expressing G225 or homologs. The observation of the reduced anthocyanin phenotype provides evidence that the crop homolog genes are active in the same pathway as G225. Further studies will be conducted to identify transgenic maize plants having improved nutrient utilization (nitrogen and/or phosphorus), drought tolerance and/or increased seed oil.

Preliminary analysis of transgenic rice plants expressing SEQ ID NO:2 indicates that the plants have improved growth under low nitrogen conditions and enhanced drought tolerance.

Preliminary analysis of transgenic Brassica plants expressing SEQ ID NO:2 resulted in identification of transgenic plants having increased seed oil levels.

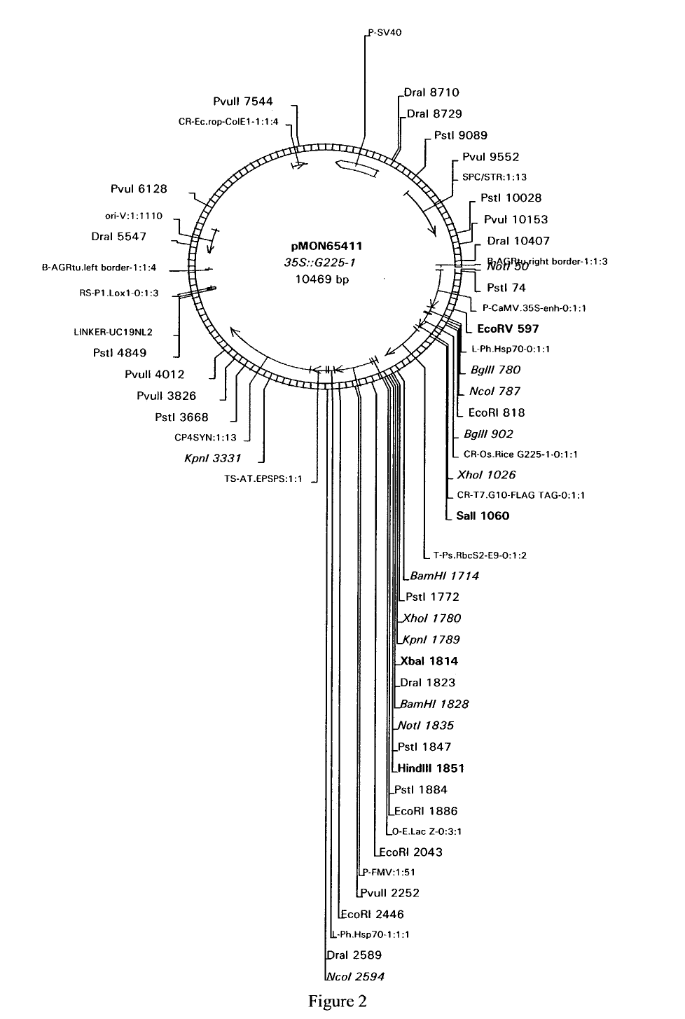

Transgenic Brassica plants expressing SEQ ID NO: 10 (rice homolog of G225) are generated. A construct for expression of the rice homolog is prepared as follows. A 1706 bp fragment, containing the promoter for the 35S RNA from CaMV with a duplication of the −90 to −300 region, the petunia hsp70 5′ untranslated leader, the coding region of a rice homolog of G225 (SEQ ID NO: 10), and 3′ end of pea rbcS E9 gene was obtained as a SmaI fragment and ligated into a PmeI digested Agrobacterium transformation vector containing a nopaline T-DNA right border sequence and octopine T-DNA left border sequence, with a 35S promoter from the Figwort Mosaic Virus (FMV) between the two T-DNA borders, proceeded by a recognition sequence for cre recombinase, driving the expression of a chimeric EPSP synthase gene containing a chloroplast targeting sequence from the Arabidopsis EPSP synthase gene (gi:16272) linked to a synthetic EPSP synthase coding region (U.S. Pat. No. 5,633,435 Barry, G. F. et al.) and the 3′ untranslated region from the pea rbcS E9 gene followed by a recognition site for cre recombinase. The resulting plasmid was designated pMON65411 (FIG. 2). DNA sequence analysis confirmed the integrity of the cloning junctions.

The vector pMON65411 is introduced into Agrobacterium tumefaciens strain ABI for transformation into Brassica napus. Canola plants are transformed using the protocol described by Moloney and Radke in U.S. Pat. No. 5,720,871. Briefly, seeds of Brassica napus cv Ebony are planted in 2 inch pots containing Metro Mix 350 (The Scotts Company, Columbus, Ohio). The plants are grown in a growth chamber at 24° C., and a 16/8 hour photoperiod, with light intensity of 400 μEm⁻² sec⁻¹ (HID lamps). After 2½ weeks, the plants are transplanted into 6 inch pots and grown in a growth chamber at 15/10° C. day/night temperature, 16/8 hour photoperiod, light intensity of 800 μm⁻² sec¹ (HID lamps).

Four terminal internodes from plants just prior to bolting or in the process of bolting but before flowering are removed and surface sterilized in 70% v/v ethanol for 1 minute, 2% w/v sodium hypochlorite for 20 minutes and rinsing 3 times with sterile deionized water. Six to seven stem segments are cut into 5 mm discs, maintaining orientation of basal end.

The Agrobacterium culture used to transform Canola is grown overnight on a rotator shaker at 24° C. in 2 mls of Luria Broth, LB, (10% bacto-tryptone, 5% yeast extract, and 10% NaCl) containing 50 mg/l kanamycin, 24 mg/l chloramphenicol and 100 mg/l spectinomycin. A 1:10 dilution is made in MS media (Murashige and Skoog Physiol. Plant, 15:473-497, (1962)) giving approximately 9×10⁸ cells per ml. The stem discs (explants) are inoculated with 1.0 ml of Agrobacterium and the excess is aspirated from the explants.

The explants are placed basal side down in petri plates containing media comprising 1/10 MS salts, B5 vitamins, 3% sucrose, 0.8% agar, pH 5.7, 1.0 mg/l 6-benzyladenine (BA). The plates are layered with 1.5 ml of media containing MS salts, B5 vitamins, 3% sucrose, pH 5.7, 4.0 mg/l p-chlorophenoxyacetic acid, 0.005 mg/l kinetin and covered with sterile filter paper.

Following a 2 to 3 day co-culture, the explants are transferred to deep dish petri plates containing MS salts, B5 vitamins, 3% sucrose, 0.8% agar, pH 5.7, 1 mg/i BA, 500 mg/l carbenicillin, 50 mg/l cefotaxime, 200 mg/l kanamycin or 175 mg/l gentamycin for selection. Seven explants are placed on each plate. After 3 weeks they are transferred to fresh media, 5 explants per plate. The explants are cultured in a growth room at 25° C., continuous light (Cool White).

The transformed plants are grown in a growth chamber at 22° C. in a 16-8 hours light-dark cycle with light intensity of 220 μEm ⁻²s⁻¹ for several weeks before transferring to the greenhouse. The plants are then grown in greenhouse conditions until maturity. The resulting mature R1 seeds are collected for analysis. Plants were maintained in a greenhouse under standard conditions. Mature seed is collected and analyzed for oil and protein content by NIR.

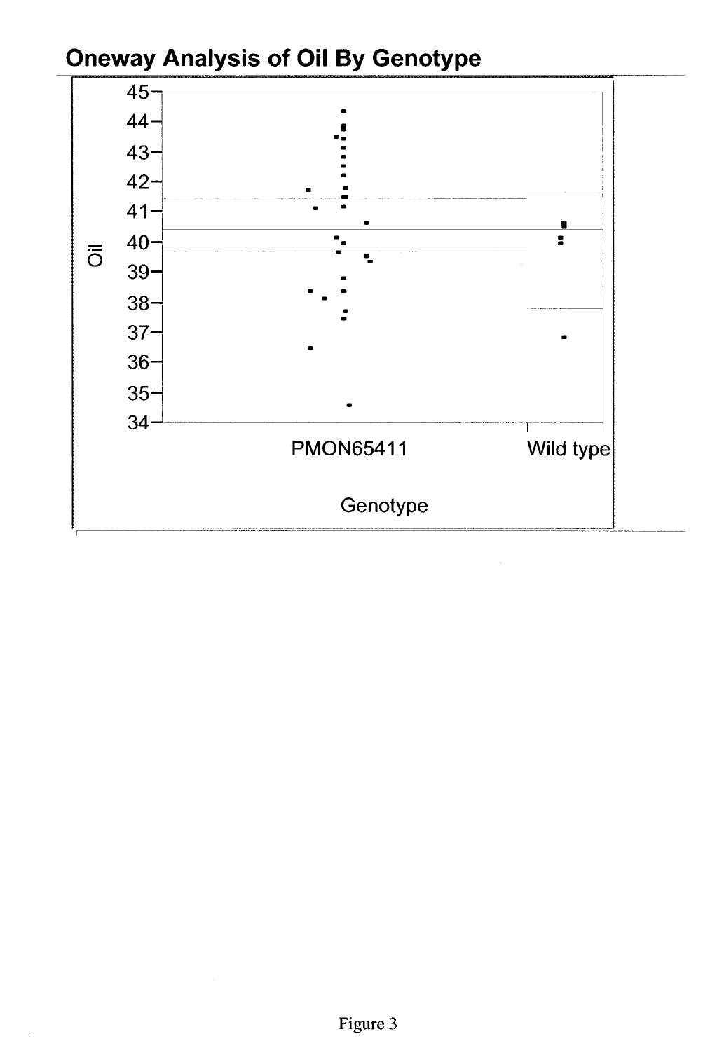

Oil levels in seeds of Canola plants transformed with pMON65411 are compared to those in seeds of non-transformed control plants of the same variety. Results are shown in FIG. 3. Percent oil in pools of seed harvested from single plants are plotted. The grand mean of both genotypes is indicated by the solid bar at ˜40.3. The confidence intervals, for each genotype, at a=0.01 are between the upper and lower broken lines. A number of events transformed with pMON65411 exceed the confidence intervals for high oil, while only four lines are below, indicating that ectopic and/or over expression of a rice G225 homolog can increase oil levels in transgenic canola.

All publications and patent applications cited herein are incorporated by reference in their entirely to the same extent as if each individual publication or patent application was specifically and individually indicated to be incorporated by reference.

We claim:

1. A recombinant DNA construct comprising a nucleic acid molecule which encodes a myb domain polypeptide molecule comprising an amino acid sequence selected from the group consisting of SEQ ID NO:16 through SEQ ID NO: 27.

2. A DNA construct of claim 1 comprising a root expressing promoter.

3. A DNA construct of claim 2 wherein said promoter is selected from the group consisting of a constitutive promoter, a drought inducible promoter mad a root epidermis expressing promoter.

4. A transgenic seed for producing a hybrid crop plant wherein the genome of said seed comprises an exogenous DNA construct which expresses in roots a myb domain polypeptide molecule wherein said myb domain consists of one or more copies of an R2 myb domain region from a plant transcription factor.

5. A transgenic seed of claim 4 but wherein said myb domain polypeptide molecule comprises an amino acid sequence selected from the group consisting of SEQ ID NO: 13 through SEQ ID NO: 27.

6. A transgenic seed of claim 5 wherein said crop plant is a monocot selected from the group consisting of maize and wheat.

7. A transgenic seed according to claim 4 wherein said myb domain polypeptide molecule is over expressed in the roots of plants grown from said seed

8. A transgenic seed for producing a crop plant wherein the genome of said seed comprises an exogenous DNA construct which expresses in roots a myb domain polypeptide molecule comprising an amino acid sequence selected from the group consisting of SEQ ID NO: 16 through SEQ ID NO: 27.

9. A transgenic seed of claim 8 wherein said crop plant is a dicot selected from the group consisting of soybean, canola and cotton.

10. A transgenic seed of claim 8 wherein said nucleic acid molecule is an endogenous plant gene which is over expressed.

11. A transgenic seed for producing a crop plant and comprising a DNA construct which expresses a nucleic acid molecule in an antisense direction which suppresses the expression of a transcription factor which regulates the root hair development activity of a myb domain polypeptide molecule comprising an amino acid sequence selected from the group consisting of SEQ ID NO: 13 through 27.

12. A transgenic seed according to claim 11 wherein said transcription factor is expressed by the werewolf gene.

13. A transgenic seed for producing a crop plant and comprising a DNA construct which expresses a double stranded RNA molecule which suppresses the expression of a transcription factor which regulates the root hair development activity of a myb domain polypeptide molecule comprising an amino acid sequence selected from the group consisting of SEQ ID NO: 13 through 27.

14. A method for improving the yield of a crop plant grown in a nutrient deficient environment for the wild type of said crop plant wherein said nutrient is selected from the group consisting of one or more of phosphorus and water, said method comprising growing a transgenic variety of said crop plant in said nutrient deficient environment wherein said plant has an exogenous DNA construct which expresses in roots a myb domain-containing polypeptide molecule wherein said myb domain consists of one or more copies of an R2 myb domain region from a plant transcription factor.

15. A method according to claim 14 wherein said myb domain-containing polypeptide comprises an amino acid sequence selected from the group consisting of SEQ ID NO: 13 through SEQ ID NO: 27.

16. A method according to claim 15 wherein said myb domain-containing polypeptide is an endogenous plant gene which is over expressed.

17. A method according to claim 16 wherein said crop plant is a monocot selected from the group consisting of maize, rice and wheat

18. A method according to claim 16 wherein said crop plant is a dicot selected from the group consisting of soybean, canola and cotton.

19. A method according to claim 15 wherein said plant comprises a DNA construct which expresses an RNA molecule which suppresses the expression of a transcription factor which regulates the root hair development activity of said myb domain polypeptide.

20. A method for improving the yield of a crop plant grown in a nitrogen deficient environment for the wild type of said crop, said method comprising growing a transgenic variety of said crop plant in a nitrogen deficient environment wherein said plant has an exogenous DNA construct which expresses in roots a myb domain-containing polypeptide molecule comprising an amino acid sequence selected from the group consisting of SEQ ID NO: 15 through SEQ ID NO: 27.

21. A method according to claim 20 wherein said myb domain-containing polypeptide is an endogenous plant gene which is over expressed.

22. A method according to claim 20 wherein said crop plant is a monocot selected from the group consisting of maize, rice and wheat

23. A method according to claim 20 wherein said crop plant is a dicot selected from the group consisting of soybean, canola and cotton.

24. A method according to claim 20 wherein said plant comprises a DNA construct which expresses an RNA molecule which suppresses the expression of a transcription factor which regulates the root hair development activity of said myb domain polypeptide.

25. A method for improving the oil yield of a crop plant as compared to wild type of said crop plant, said method comprising growing a transgenic variety of said crop plant having has an exogenous DNA construct which expresses in roots a myb domain-containing polypeptide wherein said myb domain consists of one or more copies of an R2 myb domain region from a plant transcription factor.

26. A method according to claim 25 wherein said myb domain-containing polypeptide comprises an amino acid sequence selected from the group consisting of SEQ ID NO: 13 through SEQ ID NO: 27.

27. A method according to claim 26 wherein said myb domain-containing polypeptide is an endogenous plant gene which is over expressed.

28. A method according to claim 26 wherein said crop plant is selected from the group consisting of maize, soybean, canola and cotton.

29. A method according to claim 26 wherein said plant comprises a DNA construct which expresses an RNA molecule which suppresses the expression of a transcription factor which regulates the root hair development activity of said myb domain polypeptide.
